(19) United States
(12) Patent Application Publication
(10) Pub. No.: US 2026/0000273 A1
(43) Pub. Date: Jan. 1, 2026

(54) ENDOSCOPE

(71) Applicant: LG ELECTRONICS INC., Seoul (KR)

(72) Inventors: Changkyu HWANG, Seoul (KR); Eujin KIM, Seoul (KR); Soonbeom KIM, Seoul (KR)

(73) Assignee: LG ELECTRONICS INC., Seoul (KR)

(21) Appl. No.: 19/250,964

(22) Filed: Jun. 26, 2025

(30) Foreign Application Priority Data
Jun. 26, 2024 (KR) 10-2024-0083497

(51) Int. Cl. A61B 1/00; A61B 1/005; A61B 1/05; A61B 1/06

(52) U.S. Cl. CPC A61B 1/00094; A61B 1/00103; A61B 1/0052; A61B 1/053; A61B 1/06

(57) ABSTRACT

The present disclosure relates to an endoscope including a disposable part having a lighting/imaging part and an insertion pipe, a reusable part detachably coupled to the disposable part, having a manipulation knob, and configured to bend the insertion pipe by means of a manipulation of the manipulation knob, and a disposable suction part detachably coupled to the reusable part and configured to accommodate therein a channel, in which an operator may separate the disposable suction part from the reusable part and separately discard the disposable suction part, thereby improving the hygiene of the reusable part.

### CROSS-REFERENCE TO RELATED APPLICATION

The present application claims priority to Korean Patent Application No. 10-2024-0083497, filed on Jun. 26, 2024, the entire contents of which are incorporated herein for all purposes by this reference.

### BACKGROUND OF THE DISCLOSURE

### Field of the Disclosure

The present disclosure relates to an endoscope, and more particularly, to an endoscope in which a disposable part and a reusable part may be detachably provided, and the disposable part may be used and replaced.

### Description of the Related Art

A medical endoscope relates to a device configured to be inserted directly into internal tissue of a human body, which is a target to be subjected to examinations or procedures, and used to identify states of diseased parts or perform procedures.

A general endoscope has an articulated assembly disposed at an end of a flexible insertion tube, and the articulated assembly enables an insertion direction to be adjusted during a process of inserting the endoscope into a human body. A leading end body, which is equipped with an image sensor and a light source, is provided at an end of the articulated assembly. A handle body may be provided at the other end of the insertion tube. The handle body is connected to a non-illustrated image processing device through a separate cable and a separate connector.

Dials and buttons, which are used to adjust a direction of the articulated assembly and perform the procedure, are disposed on the handle body.

The dials may be provided as a pair of dials and used to adjust a traveling direction of the leading end body upward, downward, leftward, and rightward with respect to a longitudinal direction of the insertion tube. A sprocket and a chain are provided in the handle body and convert a rotational motion of the dial into a linear motion. The chain is connected to an end of the articulated assembly through a separate wire. As a result, the rotational motion of the dial is converted into the linear motion of the wire by means of the sprocket and the chain, such that the articulated assembly is bent by the linear motion of the wire. Therefore, an operator of the endoscope may adjust the traveling direction of the leading end body during the process of inserting the insertion tube into the human body.

In this case, because the endoscope is inserted into the human body, thorough hygiene management is required, and the endoscope needs to be necessarily washed/disinfected before and after treatment or surgery. In some instances, it may be impossible or undesirable to reuse parts, which are actually inserted into the human body, i.e., the insertion tube and the articulated assembly. In this case, because the entire endoscope in the related art is integrated, there occurs a problem in that the entire endoscope needs to be replaced. In addition, even in case that the part of the endoscope may be reused, the washing and disinfection operations may be conveniently performed in case that only some parts of the endoscope may be separated without disassembling the entire endoscope.

In this regard, U.S. Patent Application Publication No. US2014/0142389A discloses an endoscope in which an insertion portion and a manipulation portion are detachably coupled.

However, in the case of the endoscope, all channels are provided in the manipulation portion that may be used multiple times, which causes a limitation in that the manipulation portion may be contaminated in case that contaminants leak from the replaceable insertion portion.

In addition, because the plurality of channels of the endoscope are disposed adjacent to one another, contaminants are introduced into the adjacent channel when the insertion portion and the manipulation portion are separated, which may cause an inconvenience of having to wash the entire manipulation portion.

### SUMMARY OF THE DISCLOSURE

The present disclosure is proposed to solve these problems of the endoscope in the related art and aims to provide an endoscope having a disposable part and a reusable part that may be easily detached by an operator.

The present disclosure also aims to provide an endoscope in which a used biopsy channel may be separately separated from a reusable part and discarded after a disposable part is used.

The present disclosure also aims to provide an endoscope capable of preventing contaminants, which remain in a disposable part, from being introduced into a reusable part after the disposable part is used.

In order to achieve the above-mentioned objects, the present disclosure provides an endoscope including: a disposable part having a lighting/imaging part and an insertion pipe; a reusable part detachably coupled to the disposable part, having a manipulation knob, and configured to bend the insertion pipe by means of a manipulation of the manipulation knob; and a disposable suction part detachably coupled to the reusable part and configured to accommodate therein a channel.

In this case, the disposable suction part may have a suction valve configured to open or close the channel.

The channel may include: a biopsy channel having one side connected to the suction valve, and the other side detachably connected to the disposable part; and a suction channel configured to connect the suction valve and a suction pump.

Meanwhile, the reusable part may accommodate a supply channel in which air or liquid flows.

In addition, the disposable suction part may include: a first suction part body having one longitudinal side detachably coupled to the disposable part; and a second suction part body extending in a direction intersecting a longitudinal direction of the first suction part body.

Meanwhile, the disposable suction part may further include a suction part handle protruding from an outer surface of the second suction part body.

Meanwhile, the disposable part may be coupled in a state in which the disposable suction part and the reusable part are coupled.

Meanwhile, the disposable suction part and the disposable part may be integrated.

In this case, the disposable suction part and the disposable part may be connected to be elastically transformable.

Meanwhile, the reusable part may include a grip guide portion provided on an outer surface and configured to guide a grip position.

Meanwhile, in order to achieve the above-mentioned objects, the present disclosure provides a component for an endoscope that may be detachably coupled to a reusable part having a manipulation knob.

In this case, the component may include: a first suction part body configured to accommodate therein a biopsy channel; and a second suction part body configured to accommodate therein a suction channel and extending in a direction intersecting a longitudinal direction of the first suction part body.

In addition, the component for an endoscope according to the present disclosure may further include: a suction part handle protruding from an outer surface of the second suction part body and configured to be gripped.

In addition, the component for an endoscope according to the present disclosure may further include: a suction valve configured to operate to allow the biopsy channel and the suction channel to communicate with each other.

In addition, the component for an endoscope according to the present disclosure may further include: a disposable part having a lighting/imaging part and an insertion pipe, in which the first suction part body integrated with the disposable part.

In this case, the first suction part body may be made of an elastic material.

### BRIEF DESCRIPTION OF THE DRAWINGS

FIG. 1 is a perspective view for explaining an endoscope according to an embodiment of the present disclosure.

FIG. 2 is a perspective view illustrating the endoscope according to the embodiment of the present disclosure when viewed at another angle.

FIG. 3 is an exploded perspective view illustrating a state in which the endoscope according to the embodiment of the present disclosure is disassembled.

FIG. 4 is a cross-sectional view of a reusable part of the endoscope according to the embodiment of the present disclosure.

FIG. 5 is a bottom plan view of the reusable part of the endoscope according to the embodiment of the present disclosure.

FIG. 6 is a perspective view for explaining a disposable suction part of the endoscope according to the embodiment of the present disclosure.

FIG. 7 is a view for explaining a channel of the endoscope according to the embodiment of the present disclosure.

FIG. 8 is a side view for explaining an endoscope according to a second embodiment of the present disclosure.

FIG. 9 is a cross-sectional view for explaining a disposable part of the endoscope according to the embodiment of the present disclosure.

FIG. 10 is a top plan view for explaining connection between a wire reel and a wire of the disposable part according to the embodiment of the present disclosure.

FIG. 11 is a view for explaining a channel in the disposable part of the endoscope according to the embodiment of the present disclosure.

FIG. 12 is a perspective view for explaining a lighting/imaging part of the disposable part according to the embodiment of the present disclosure.

FIG. 13 is a perspective view for explaining an endoscope according to a third embodiment of the present disclosure.

FIG. 14 is a perspective view for explaining a reusable part of the endoscope according to the third embodiment of the present disclosure.

FIG. 15 is a perspective view for explaining a disposable suction part of the endoscope according to the third embodiment of the present disclosure.

### DETAILED DESCRIPTION OF THE DISCLOSURE

Hereinafter, exemplary embodiments of the present disclosure will be described in detail with reference to the accompanying drawings.

The present disclosure may be variously modified and may have various embodiments, and particular embodiments illustrated in the drawings will be specifically described below. The description of the embodiments is not intended to limit the present disclosure to the particular embodiments, but it should be interpreted that the present disclosure is to cover all modifications, equivalents and alternatives falling within the spirit and technical scope of the present disclosure.

In the description of the present disclosure, the terms such as “first” and “second” may be used to describe various constituent elements, but the constituent elements may not be limited by the terms. These terms are used only to distinguish one constituent element from another constituent element. For example, a first component may be named a second component, and similarly, the second component may also be named the first component, without departing from the scope of the present disclosure.

The term “and/or” may include any and all combinations of a plurality of the related and listed items.

When one constituent element is described as being “coupled” or “connected” to another constituent element, it should be understood that one constituent element can be coupled or connected directly to another constituent element, and an intervening constituent element can also be present between the constituent elements. When one constituent element is described as being “coupled directly to” or “connected directly to” another constituent element, it should be understood that no intervening constituent element is present between the constituent elements.

The terminology used herein is used for the purpose of describing particular embodiments only and is not intended to limit the present disclosure. Singular expressions may include plural expressions unless clearly described as different meanings in the context.

The terms “comprises,” “comprising,” “includes,” “including,” “containing,” “has,” “having” or other variations thereof are inclusive and therefore specify the presence of stated features, integers, steps, operations, elements, components, and/or combinations thereof, but do not preclude the presence or addition of one or more other features, integers, steps, operations, elements, components, and/or combinations thereof.

Unless otherwise defined, all terms used herein, including technical or scientific terms, may have the same meaning as commonly understood by those skilled in the art to which the present disclosure pertains. The terms such as those defined in a commonly used dictionary may be interpreted as having meanings consistent with meanings in the context of related technologies and may not be interpreted as ideal or excessively formal meanings unless explicitly defined in the present application.

Further, the following embodiments are provided to more completely explain the present disclosure to those skilled in the art, and shapes and sizes of elements illustrated in the drawings may be exaggerated for a more apparent description.

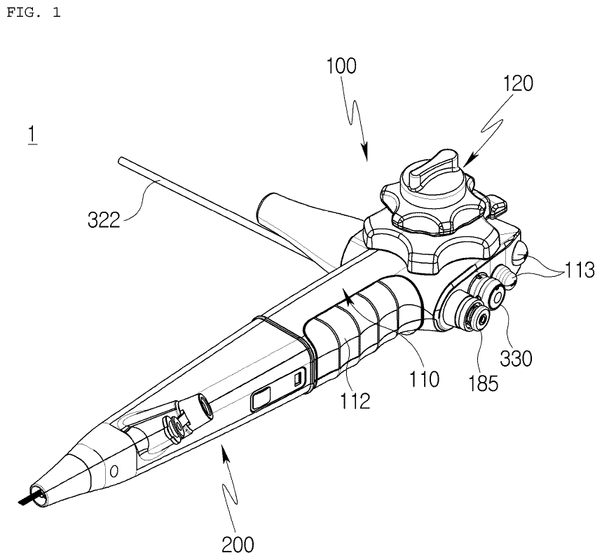

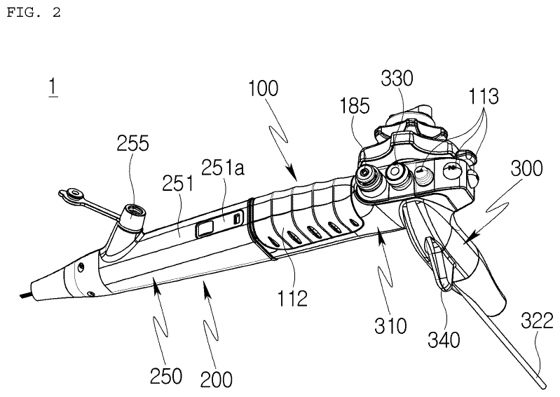

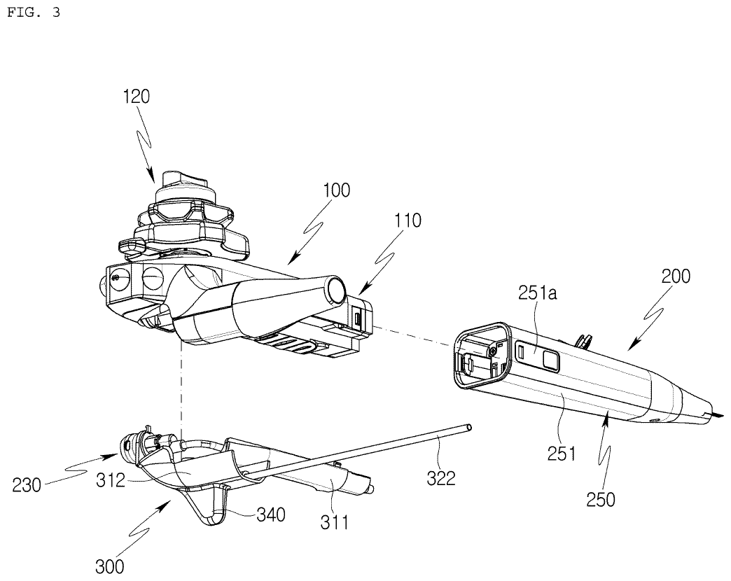

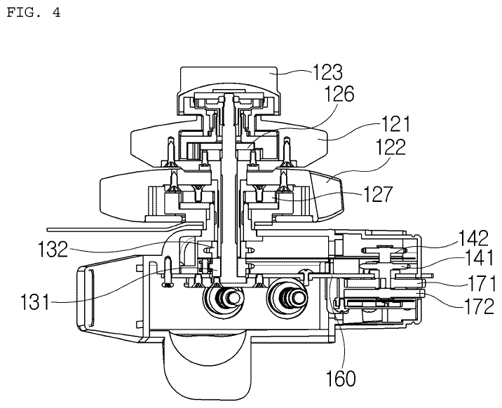

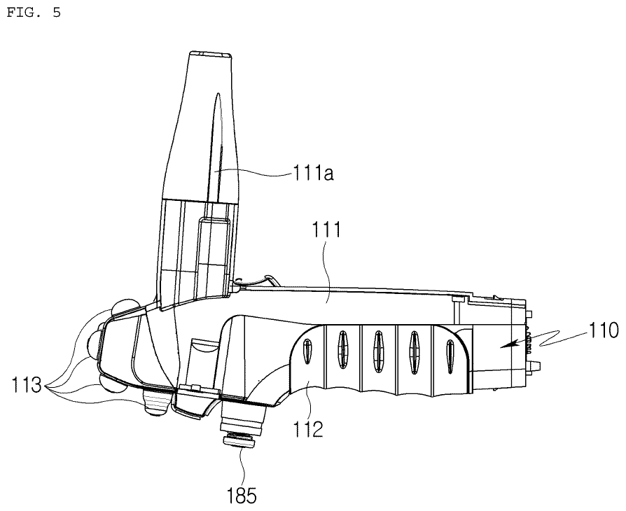

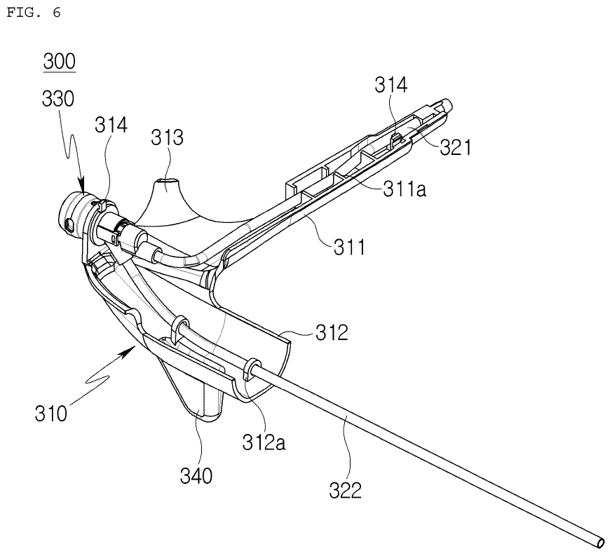

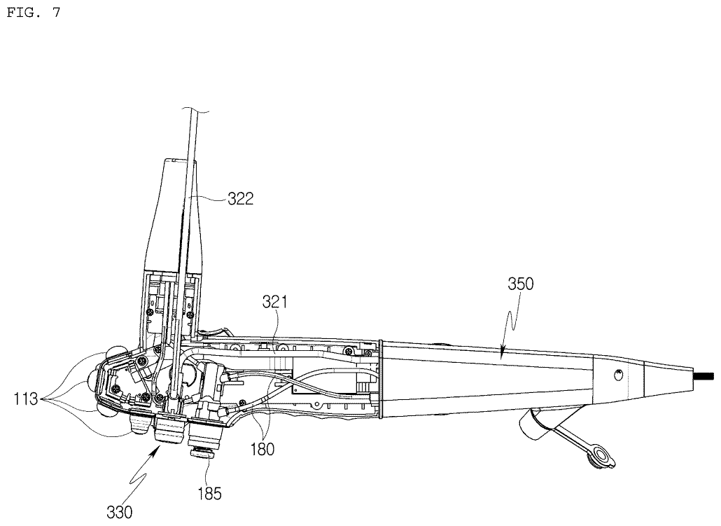

FIG. 1 is a perspective view for explaining an endoscope according to an embodiment of the present disclosure, FIG. 2 is a perspective view illustrating the endoscope according to the embodiment of the present disclosure when viewed at another angle, FIG. 3 is an exploded perspective view illustrating a state in which the endoscope according to the embodiment of the present disclosure is disassembled, FIG. 4 is a cross-sectional view of a reusable part of the endoscope according to the embodiment of the present disclosure, FIG. 5 is a bottom plan view of the reusable part of the endoscope according to the embodiment of the present disclosure, FIG. 6 is a perspective view for explaining a disposable suction part of the endoscope according to the embodiment of the present disclosure, and FIG. 7 is a view for explaining a channel of the endoscope according to the embodiment of the present disclosure.

With reference to FIGS. 1 to 7, an endoscope 1 according to the embodiment of the present disclosure may be a disposable endoscope. Specifically, the endoscope 1 according to the embodiment of the present disclosure includes a reusable part 100 and a disposable part 200. In this case, the reusable part 100 and the disposable part 200 may be detachably coupled to each other.

The reusable part 100 is provided to be manipulated by an operator. The reusable part 100 may be provided to be gripped and manipulated by the operator. The operator may manipulate the reusable part 100 to bend the disposable part 200.

The disposable part 200 is provided to be inserted into a human body by the manipulation of the reusable part 100 and capture images of the inside of the human body. The disposable part 200 may be inserted into the human body and provided to be bendable by the manipulation of the reusable part 100, such that the disposable part 200 may move along an internal structure of the human body, illuminate the inside of the human body, and capture images of the inside of the human body.

The endoscope 1 according to the embodiment of the present disclosure may further include a connector adapter and a universal cord.

Specifically, the endoscope 1 may include a connector adapter configured to be connected to an external device, and the connector adapter may be connected to the reusable part 100 by the universal cord. The external device connected to the connector adapter may be an image processing device. Therefore, the image captured by the disposable part 200 may be displayed on an external image processing device. Therefore, the operator may manipulate the reusable part 100 while observing the image processing device that displays the image captured by the disposable part 200.

The reusable part 100 includes a reusable part housing 110, a manipulation knob 120, a power transmission part, and a reusable part plate.

The reusable part housing 110 is provided to be gripped by a practitioner. For example, the reusable part housing 110 may be formed in a hollow container shape, and a grip guide portion 112 may be formed on an outer surface of the reusable part housing 110. In this case, the grip guide portion 112 may have a plurality of bent grooves to guide a position of a hand of the practitioner (operator). In addition, a plurality of protrusions or grooves may be formed on the grip guide portion 112 to prevent the hand of the practitioner from slipping.

The reusable part housing 110 may have the manipulation knob 120, a plurality of valves 185 and 330, and a plurality of buttons 113. For example, the manipulation knob 120 may be rotatably coupled to one longitudinal side of the reusable part housing 110, and the plurality of valves 185 and 330 and the plurality of buttons 113 may be disposed around the manipulation knob 120. In this case, based on the grip guide portion 112, the disposable part 200 may be disposed in a direction opposite to the manipulation knob 120, the plurality of valves 185 and 330, and the plurality of buttons 113. That is, when the manipulation knob 120, the plurality of valves 185 and 330, and the plurality of buttons 113 are disposed at one longitudinal side of the reusable part housing 110, the disposable part 200 may be coupled to the other longitudinal side of the reusable part housing 110.

With this t configuration, in a state in which the practitioner grasps the grip guide portion 112 with his/her palm, the practitioner may push the valves 185 and 330 and the buttons 113 and easily rotate the manipulation knob 120 with the hand opposite to the hand that grasps the reusable part housing 110.

Meanwhile, the reusable part housing 110 may accommodate therein the power transmission part configured to operate the disposable part 200.

The power transmission part may rotate in conjunction with the manipulation knob 120 and transmit a rotational force of the manipulation knob 120. Power transmission means with various shapes may be applied to the power transmission part. For example, the power transmission part may include a plurality of sprockets and a chain. In another example, the power transmission part may include a plurality of timing gears and a timing belt. In still another example, the power transmission part may have a plurality of gears configured to engage with one another.

Hereinafter, the structure including the sprockets and the chain will be described.

Power transmission parts 130, 140, and 150 according to the embodiment of the present disclosure may include a driving sprocket 130, a driven sprocket 140, and a manipulation chain 150.

In this case, the reusable part housing 110 may accommodate therein the driving sprocket 130 configured to operate in conjunction with the rotation of the manipulation knob 120, the driven sprocket 140 configured to rotate in conjunction with the driving sprocket 130, and the manipulation chain 150 configured to connect the driving sprocket 130 and the driven sprocket 140.

For example, the driving sprocket 130 may be disposed at one longitudinal side in the reusable part housing 110, and the driven sprocket 140 may be disposed at the other longitudinal side in the reusable part housing 110. In addition, the manipulation chain 150 may be disposed in the longitudinal direction in the reusable part housing 110.

Meanwhile, the reusable part housing 110 may accommodate supply channels 180.

The manipulation knob 120 may control the disposable part 200 while being rotated by the manipulation of the user. When the manipulation knob 120 is rotated by the user's manipulation, the sprockets 130 and 140 may rotate in conjunction with the rotation of the manipulation knob 120.

The manipulation knob 120 may include two or more knobs. For example, the manipulation knob 120 may be provided in a shape in which a first knob 121 and a second knob 122 are stacked in an upward/downward direction. The first knob 121 and the second knob 122 are respectively connected to a first driving sprocket 131 and a second driving sprocket 132 that will be described below, and the first knob 121 and the second knob 122 may be configured to rotate independently of each other. The first knob 121 and the second knob 122 may each have a shape in which a plurality of teeth protrude so that the practitioner may easily grip and rotate the first knob 121 and the second knob 122.

Meanwhile, the first knob 121 may rotate independently while the second knob 122 is kept stopped. Likewise, the second knob 122 may rotate independently while the first knob 121 is kept stopped. The first knob 121 controls upward and downward movements of a lighting/imaging part 210. That is, when the first knob 121 rotates clock or counterclockwise, an end of the lighting/imaging part 210 is bent upward or downward. The second knob 122 controls leftward and rightward movements of the lighting/imaging part 210. That is, when the second knob 122 rotates clock or counterclockwise, the end of the lighting/imaging part 210 is bent leftward or rightward.

Meanwhile, in another embodiment, the first knob 121 may control the leftward and rightward movements of the lighting/imaging part 210, and the second knob 122 may control the upward and downward movements of the lighting/imaging part 210.

As described above, the rotation direction of the lighting/imaging part 210 may be arbitrarily adjusted by manipulating the first knob 121 and the second knob 122. According to the embodiment, it may be necessary to fix a state in which the lighting/imaging part 210 is bent at a particular angle. To this end, a fixing knob 123 is provided above the second knob 122. When the fixing knob 123 is rotated, the first driving sprocket 131 and the second driving sprocket 132 are fixed so as not to rotate, such that the end of the lighting/imaging part 210 may also be kept fixed.

The first driving sprocket 131 and the second driving sprocket 132 are mounted below the first knob 121 and the second knob 122 and configured to be rotatable in conjunction with the rotations of the first and second knobs 121 and 122. Further, the manipulation chain 150 is provided to engage with the driving sprocket 130 and configured to move. The manipulation chain 150 may perform the upward and downward movements and the leftward and rightward movements of the lighting/imaging part 210, and the manipulation chain 150 and the lighting/imaging part 210 may move independently of each other.

Specifically, a first knob shaft 126 may be coupled in the first knob 121, and the first knob 121 and the first knob shaft 126 may rotate together. In addition, a second knob shaft 127 is coupled in the second knob 122, and the second knob 122 and the second knob shaft 127 may rotate together. In this case, the first knob shaft 126 may be coupled to the first driving sprocket 131. Further, the second knob shaft 127 may be coupled to the second driving sprocket 132.

Meanwhile, the first knob 121 may be disposed above the second knob 122 based on a state in which the reusable part housing 110 is placed to be closer to a ground surface than the manipulation knob 120. In addition, the fixing knob 123 may be disposed above the first knob 121.

Meanwhile, the power transmission parts 130, 140, and 150 may include first power transmission parts 131, 141, and 151 and second power transmission parts 132, 142, and 152. In this case, the second power transmission parts 132, 142, and 152 may be disposed above the first power transmission parts 131, 141, and 151. Specifically, the second driving sprocket 132 may be disposed above the first driving sprocket 131. That is, the second knob 122 and the second driving sprocket 132 may be disposed between the first knob 121 and the first driving sprocket 131.

The manipulation chain 150 may connect the driving sprocket 130 and the driven sprocket 140. The manipulation chain 150 may transmit a rotational force of the driving sprocket 130 to the driven sprocket 140. When the driving sprocket 130 rotates by means of the manipulation chain 150, the driven sprocket 140 may rotate in conjunction with the rotation of the driving sprocket 130.

The manipulation chain 150 include may a first manipulation chain 151 and a second manipulation chain 152. The first manipulation chain 151 may connect the first driving sprocket 131 and a second driven sprocket 141. The second manipulation chain 152 may connect the second driving sprocket 132 and a second driven sprocket 142.

In this case, the second driven sprocket 142 may be disposed above the first driven sprocket 141.

Therefore, when the first knob 121 is rotated by the user's manipulation, the first driving sprocket 131 and the first driven sprocket 141 may rotate in conjunction with the rotation of the first knob 121. In addition, when the second knob 122 is rotated by the user's manipulation, the second driving sprocket 132 and the second driven sprocket 142 may rotate in conjunction with the rotation of the second knob 122.

Meanwhile, the reusable part 100 may include a reusable part plate 160. The reusable part plate 160 may divide the inside of the reusable part housing 110 in the upward/downward direction. For example, the reusable part plate 160 may be formed in a flat plate shape and disposed in the reusable part housing 110. In this case, the driving sprocket 130, the driven sprocket 140, and the manipulation chain 150 may be disposed above the reusable part housing 110. In addition, a driving gear 170 and the channels 180 may be disposed below the reusable part housing 110. The driving gear 170 may rotate in conjunction with the rotations of the sprockets 130 and 140. Specifically, the driving gear 170 may receive driving power from the driven sprocket 140. In this case, the first driven sprocket 141 may be coupled to a first driving gear 171.

Specifically, the first driven sprocket 141 may be spline-coupled to the first driving gear 171. Alternatively, the first driven sprocket 141 may be key-coupled to the first driving gear 171. For example, the first driven sprocket 141 may include a sprocket portion coupled to the manipulation chain 150, and a coupling shaft portion extending axially downward from the sprocket portion and coupled to the first driving gear 171. In this case, a key portion may be formed on an outer peripheral surface of the coupling shaft portion and spline-coupled to the first driving gear 171. Therefore, when the first driven sprocket 141 rotates, the first driving gear 171 may also rotate in conjunction with the rotation of the first driven sprocket 141.

Meanwhile, the second driven sprocket 142 may be spline-coupled to a second driving gear 172. Alternatively, the second driven sprocket 142 may be key-coupled to the second driving gear 172. For example, the second driven sprocket 142 may include a sprocket portion coupled to the manipulation chain 150, and a shaft coupling hole formed at a radial center of the sprocket portion. In this case, the shaft coupling hole may be formed as a hole having a shape other than a circular shape so that the shaft coupling hole is key-coupled to a connection shaft 173. In addition, the second driving gear 172 may include a gear portion having gear teeth, and a shaft coupling hole formed at a radial center of the gear portion. Therefore, the second driven sprocket 142 may be connected to the second driving gear 171 through the connection shaft 173. Therefore, when the second driven sprocket 142 rotates, the second driving gear 172 may also rotate in conjunction with the rotation of the second driven sprocket 142.

Meanwhile, the first driving gear 171 may be disposed above the second driving gear 172. Therefore, the first driven sprocket 141 and the first driving gear 171 may be disposed between the second driven sprocket 142 and the second driving gear 172.

Meanwhile, channels may be provided in the reusable part 100 and provide air and water to a diseased part during a procedure. For example, the channel may be the supply channel 180. In this case, the supply channels 180 may be an air supply channel and/or a water supply channel. The air supply channel and the water supply channel each have an approximately cylindrical shape and have an empty interior. The air supply channel or the water supply channel is inserted into the lighting/imaging part 210 when the lighting/imaging part 210 is coupled, such that air or water may be stably supplied to a site subjected to a procedure or examination without leaking.

Meanwhile, a supply valve 185 may be provided on the supply channels 180. The supply valve 185 may be exposed to the outside of the reusable part housing 110. When the operator operates the supply valve 185, the supply valve 185 may switch the supplies of air and water to be introduced into the disposable part 200 through the supply channels 180.

The connection terminal may be provided to be electrically connected to the lighting/imaging part 210 so that data obtained by a camera or the like provided at the end of the lighting/imaging part 210 may be transmitted to the external device. For example, the connection terminal may be an HDMI terminal configured to transmit voices and images.

The channel 180 and the connection terminal may be disposed below the manipulation chain 150. For example, the channel 180 and the connection terminal may be accommodated in the reusable part housing 110 and disposed to be farther from the manipulation knob 120 than the manipulation chain 150.

In this case, a region, in which the manipulation chain 150 moves, and a region, in which the channel 180 and the connection terminal are disposed, may be distinguished by the reusable part plate 160. The channel 180 of the reusable part 100 may be connected to a channel (not illustrated) of the disposable part 200.

The reusable part 100 includes a coupling portion 190. The coupling portion 190 may be detachably coupled to a connecting part 250 of the disposable part 200. The coupling portion 190 and coupled to a hook 251a of the connecting part 250. Specifically, the coupling portion 190 may be formed on the outer surface of the reusable part housing 110 and accommodate the hook of the connecting part 250.

Meanwhile, a guide pin may protrude from a longitudinal end surface of the coupling portion 190 and guide a position at which the coupling portion 190 is coupled to the connecting part 250. Therefore, the user may accurately couple the reusable part 100 and the disposable part 200.

Meanwhile, a gear hole may be formed in the longitudinal end surface of the coupling portion 190. The gear hole may be disposed so that at least a part of the driving gear 170 may pass through the gear hole. In this case, in a state in which the reusable part 100 is separated from the disposable part 200, at least a part of the driving gear 170 may be exposed to the outside.

In addition, a channel connection portion may be provided on the longitudinal end surface of the coupling portion 190. A longitudinal end of the channel 180 may be disposed on the channel connection portion. Therefore, when the reusable part 100 and the disposable part 200 are coupled, the channel 180 may be connected to a channel 243 of the disposable part 200.

In addition, a connector may be provided on the longitudinal end surface of the coupling portion 190 and connect the connection terminal. The connector may be provided to connect the connection terminal.

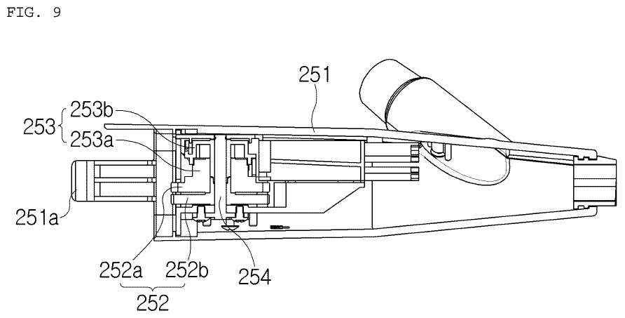

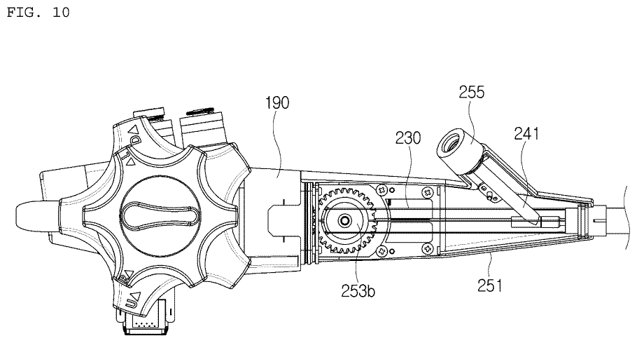

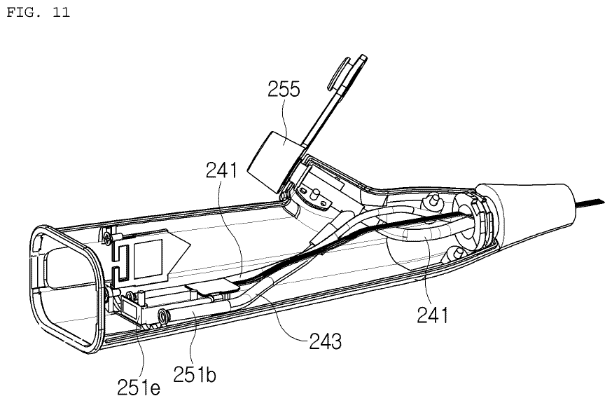

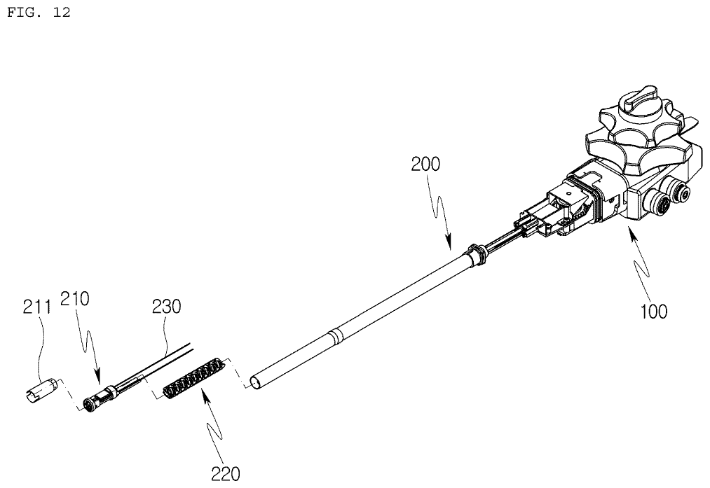

FIG. 9 is a cross-sectional view for explaining a disposable part of the endoscope according to the embodiment of the present disclosure, FIG. 10 is a top plan view for explaining connection between a wire reel and a wire of the disposable part according to the embodiment of the present disclosure, FIG. 11 is a view for explaining a channel in the disposable part of the endoscope according to the embodiment of the present disclosure, and FIG. 12 is a perspective view for explaining lighting/imaging part of the disposable part according to the embodiment of the present disclosure.

The disposable part 200 of the endoscope according to the embodiment of the present disclosure will be described below with reference to FIGS. 9 to 12.

The disposable part 200 may be detachably coupled to the reusable part 100, at least a part of the disposable part 200 may be inserted into the human body, and the disposable part 200 may capture images of the inside of the human body.

The disposable part 200 includes the lighting/imaging part 210, an insertion pipe 220, a wire 230, and the connecting part 250. In this case, the lighting/imaging part 210 is disposed at one longitudinal side of the disposable part 200, and the connecting part 250 is disposed at the other side of the disposable part 200. In addition, the lighting/imaging part 210 and the connecting part 250 may be connected to the insertion pipe 220, and at least a part of the wire 230 may pass through the connecting part 250 and the insertion pipe 220 from the connecting part 250 to the insertion pipe 220.

The lighting/imaging part 210 may be inserted into the human body, illuminate the inside of the human body with light, and capture images of the inside of the human body.

The lighting/imaging part 210 includes a lighting/imaging part body 211, a lighting module, and an image capturing module.

The lighting/imaging part body 211 is configured to define an external appearance of the lighting/imaging part 210 and be inserted into the human body. For example, the lighting module and the image capturing module may be disposed at one longitudinal side of the lighting/imaging part body 211, and the other longitudinal side of the lighting/imaging part body 211 may be opened and connected to the insertion pipe 220.

In this case, according to the embodiment, the lighting/imaging part body 211 may serve as a heat dissipation chassis.

For example, the lighting/imaging part body 211 may be a coil pipe made of a spring material and dissipate heat generated from the lighting module and/or the image capturing module. The lighting module may be coupled to an end cap. The lighting module may emit light. The lighting module may have a lighting lens capable of emitting light by receiving power. For example, the lighting lens may be a light-emitting diode (LED).

Meanwhile, in the present embodiment, the lighting lens may be provided as a plurality of lighting lenses. Specifically, in the present embodiment, an even number of lighting lenses may be provided. For example, the lighting lenses may be provided as a pair of lighting lenses disposed to be linearly symmetrical. Therefore, it is possible to prevent illuminance of an object created at a short distance from being asymmetrical and prevent gaps in lighting.

In this case, the pair of lighting lenses may be different in correlated color temperature (CCT) from each other. Therefore, it is possible to create a targeted wavelength distribution of light.

In addition, the lighting module may further include a lighting support portion configured to support the pair of lighting lenses. The lighting support portion may be formed in a flat plate shape and have a pair of leading end portions extending symmetrically and bent upward. The pair of lighting lenses may be coupled to the pair of bent extension portions.

For example, the lighting support portion may be a flexible printed circuit board (PCB). An image sensor may be mounted on the flexible PCB. Because the plurality of lighting lenses are used for the lighting part, it is possible to easily adjust the illuminance and significantly increase the amount of light emission in comparison with a halogen lamp or the like in the related art. Therefore, it is possible to further improve the clarity of images obtained by the image sensor.

Meanwhile, the image capturing module may capture images of the inside of the human body. The image capturing module may capture images of the inside of the human body by receiving power and transmit the captured image data. For example, the image capturing module may include a camera and an image processor. In this case, the camera may be disposed at one longitudinal end (leading end) of the lighting/imaging part 210, and the image processor may be coupled to the camera and process the image obtained by the camera.

Therefore, in the state in which the endoscope 1 of the present disclosure is inserted into the human body, the lighting module may emit light, and the image capturing module may capture images of the inside of the human body.

The end cap may be made of a transparent material. A plurality of channel accommodation pipes, into which the channels (not illustrated) are inserted, may be formed in the end cap. The channel accommodation pipes each have a hollow cylindrical shape and have an inner diameter corresponding to an outer diameter of the channel to be inserted. Further, an O-ring may be provided to prevent a leak of supplied water or air.

Meanwhile, the lighting/imaging part 210 may have a connection terminal. The connection terminal may be provided to correspond to the connection terminal of the reusable part 100. That is, the reusable part 100 may have a male terminal, and the disposable part 200 may have a female terminal.

The insertion pipe 220 may connect the lighting/imaging part 210 and the connecting part 250 and be bent by the manipulation of the reusable part 100.

Specifically, the insertion pipe 220 may be bent by the movement of the wire 230.

Meanwhile, a plurality of channels 240 and a terminal may be accommodated in the insertion pipe 220.

The insertion pipe 220 may include a bending portion, adapters, a coil spring, and an insertion tube. In this case, the bending portion may be disposed between the pair of adapters. One of the pair of adapters may be coupled to the lighting/imaging part 210, and the other of the pair of adapters may be coupled to the connecting part 250.

The bending portion may be configured to be bent by the movement of the wire 230.

The bending portion includes first and second bending members configured to rotate relative to one another. In this case, the bending portion may be configured such that the plurality of first bending members and the plurality of second bending members are disposed alternately. Further, an overall length of the bending portion may increase as the number of first bending members and the number of second bending members are increased. Therefore, an appropriate length of the insertion pipe 220 may be selected by adjusting the number of first bending members and the number of second bending members in accordance with a condition of use of the endoscope 1.

The first and second bending members may be provided to rotate relative to each other.

In this case, the wire 230 may pass through wire through-holes formed in the first and second bending members.

The adapters may be respectively coupled to two opposite longitudinal ends of the bending portion. In this case, the adapter disposed at one longitudinal side may be coupled to the lighting/imaging part 210, and the adapter disposed at the other longitudinal side may be coupled to the connecting part 250.

In this case, the wire 230 may pass through the wire through-holes of the adapters.

Meanwhile, the coil spring may be disposed to surround an outer peripheral surface of the bending portion. That is, the coil spring may be disposed in a shape that surrounds outer peripheral surfaces of the first and second bending members in a state in which the first and second bending members are coupled.

In contrast, two opposite longitudinal ends of the coil spring may be coupled to the adapters. The coil spring may be elastically supported by spring coupling portions of the pair of adapters. Therefore, in case that the bending portion is bent by the manipulation of the manipulation knob 120, the coil spring may apply an elastic force to restore the bending portion to an original position.

Meanwhile, the insertion tube may be disposed at the other longitudinal side of the insertion pipe 220. The insertion tube may be formed in a bendable tubular shape, and the wire 230, the channel 240, and the terminal may pass through the insertion tube. Although not illustrated, a coil pipe may be provided in an insertion tube and provide an elastic force to the insertion tube.

Meanwhile, the wire 230 may receive a manipulation force of the manipulation knob 120 through the connecting part 250 and bend the insertion pipe 220 by means of a rectilinear reciprocating motion.

One longitudinal end of the wire 230 may be coupled to the adapter or the lighting/imaging part 210, and the other longitudinal end of the wire 230 may be coupled to a wire reel 253. In this case, the wire 230 may pass through the wire through-hole of the adapter, the wire through-hole of the first bending member, and the wire through-hole of the second bending member.

Therefore, at a side at which the wire 230 is pulled, the first bending member and the second bending member may rotate relative to each other so that an interval between the first bending member and the second bending member decreases. In contrast, at a side at which the wire 230 is unwound (extended), the first bending member and the second bending member may rotate relative to each other so that the interval between the first bending member and the second bending member increases. Therefore, the first bending member and the second bending member may define a bent shape.

The disposable part 200 may have the channels 240. The channels 240 may be accommodated in the insertion pipe 220 and a connecting part housing 251. Specifically, the channels 240 may include a biopsy channel 241 and the supply channel 243.

One side of the biopsy channel 241 may be connected to the lighting/imaging part 210, and the other side of the biopsy channel 241 may be detachably connected to a disposable suction part 300. The biopsy channel 241 may provide a route along which a tool configured to collect tissue from a part of a living body or an organ moves or a collected specimen moves. Meanwhile, a part of the biopsy channel 241 may branch and communicate with a biopsy insertion port into which the tool is inserted. The biopsy insertion port may be closed by a biopsy cap 255, and the biopsy cap 255 may be removed when the tool is inserted.

The supply channel 243 may provide air and water to a diseased part during a procedure. In this case, the supply channel 243 may supply air and/or water. The supply channel 243 has an approximately cylindrical shape and has an empty interior. The supply channel 243 is inserted into the lighting/imaging part 210, such that air or water may be stably supplied to a site subjected to a procedure or examination without leaking.

The connecting part 250 is detachably coupled to the coupling portion 190 of the reusable part 100. The connecting part 250 may include the connecting part housing 251, a driven gear 252, and the wire reel 253.

One longitudinal side of the connecting part housing 251 may be coupled to the insertion pipe 220, and the other longitudinal side of the connecting part housing 251 may be detachably coupled to the coupling portion 190. In this case, one longitudinal side of the connecting part housing 251 may be coupled to the other longitudinal side of the insertion pipe 220, and the other longitudinal side of the connecting part housing 251 may be detachably coupled to the coupling portion 190.

The connecting part housing 251 may be formed in a quadrangular container shape as a whole and formed in a shape having a diameter that decreases toward one longitudinal end (leading end). As a result, one longitudinal end of the connecting part housing 251 may be formed in a shape that may be coupled to the insertion tube of the insertion pipe 220, and one longitudinal end of the connecting part housing 251 may be connected to the insertion tube of the insertion pipe 220.

The other longitudinal side of the connecting part housing 251 may be provided to engage with and be coupled to the coupling portion 190.

Specifically, at least one hook 251a may extend from the other longitudinal end of the connecting part housing 251. For example, the hooks 251a may be respectively disposed on two surfaces of the connecting part housing 251 that face each other. In this case, the hook 251a may be coupled to the coupling portion 190 formed on the outer surface of the reusable part housing 110. Specifically, the hook 251a may slide along the outer surface of the reusable part housing 110 and be accommodated in and coupled to a groove formed in the outer surface of the reusable part housing 110.

In this case, according to the embodiment, the connecting part housing 251 may further have a guide part configured to guide a position at which the connecting part housing 251 is coupled to the coupling portion 190. In this case, one guide part may be formed on a surface different from the two surfaces on which the pair of hooks 251a are respectively formed. For example, the guide part may extend from an upper surface of the connecting part housing 251. Therefore, the user may distinguish which side is an upper side and which side is a lower side when coupling the connecting part 250 and the coupling portion 190, thereby preventing erroneous assembling.

Meanwhile, a pin accommodation groove may be formed in the other longitudinal end surface of the connecting part housing 251 and accommodate a guide pin. Therefore, the user may couple the coupling portion 190 and the connecting part 250 at an exact position.

Meanwhile, a gear hole may be formed in the other longitudinal end surface of the connecting part housing 251. The gear hole may be disposed such that at least a part of the driven gear 252 passes through the gear hole. In this case, in the state in which the reusable part 100 is separated from the disposable part 200, at least a part of the driven gear 252 may be exposed to the outside.

In addition, a channel connection portion 251b may be provided on the other longitudinal end surface of the connecting part housing 251. A longitudinal end of the channel 240 may be disposed on the channel connection portion 251b. Therefore, when the reusable part 100 and the disposable part 200 are coupled, the supply channel 180 of the reusable part 100 and the supply channel 243 of the disposable part 200 may be connected.

In addition, a connector 251e capable of connecting the connection terminal may be provided on the other longitudinal end surface of the connecting part housing 251. Therefore, when the reusable part 100 and the disposable part 200 are coupled, a connector 196 of the reusable part 100 and the connector 251e of the disposable part 200 may be connected to each other.

As a result, the connecting part housing 251 and the coupling portion 190 may be fitted with each other. In this case, the driving gear 170 and the driven gear 252 may engage with each other, the supply channel 180 of the reusable part 100 and the supply channel 243 of the disposable part 200 may be connected to each other, and the terminal of the reusable part 100 and the terminal of the disposable part 200 may be connected to each other.

The driven gear 252 may receive driving power from the driving gear 170. When the reusable part 100 and the disposable part 200 are coupled, the driven gear 252 may engage with the driving gear 170. Specifically, the driven gear 252 includes a first driven gear 252a and a second driven gear 252b. In this case, when the reusable part 100 and the disposable part 200 are coupled, the first driven gear 252a may engage with the first driving gear 171, and the second driven gear 252b may engage with the second driving gear 172.

Therefore, when the first driving gear 171 rotates, the first driven gear 252a may rotate in conjunction with the rotation of the first driving gear 171. When the second driving gear 172 rotates, the second driven gear 252b may rotate in conjunction with the rotation of the second driving gear 172.

Meanwhile, the first driven gear 252a may rotate a first wire reel 253a by receiving a rotational force from the first driving gear 171. In addition, the second driven gear 252b may rotate a second wire reel 253b by receiving a rotational force from the second driving gear 172.

For example, the first driven gear 252a may be integrated with the first wire reel 253a. In this case, the first driven gear 252a and the first wire reel 253a may each have a radial central portion having a hollow shape. Therefore, an overall number of components may be reduced.

Meanwhile, the second driven gear 252b may include a gear portion having a gear teeth, and a shaft portion extending upward in an axial direction from the radial central portion of the gear portion. In this case, the shaft portion may penetrate a hollow portion of the first driven gear 252a and a hollow portion of the first wire reel 253a. Further, an axial upper end of the shaft portion may be key-coupled to the second wire reel 253b. Therefore, the second driven gear 252b and the second wire reel 253b may be coupled to each other and rotate together.

Meanwhile, the first driven gear 252a may be disposed above the second driven gear 252b. In addition, the first wire reel 253a may be disposed below the second wire reel 253b. That is, the first driven gear 252a and the first wire reel 253a may be disposed between the second driven gear 252b and the second wire reel 253b.

Meanwhile, the wire reel 253 may move the wire 230 while rotating in conjunction with the rotation of the driven gear 252. The wire reel 253 may include the first wire reel 253a and the second wire reel 253b.

In this case, the first wire reel 253a may include a reel body formed in a cylindrical or circular column shape, and a wire fixing portion disposed radially outward of the reel body and configured such that the wire 230 is fixedly coupled to the wire fixing portion. In this case, an axially lower end of the reel body may extend radially outward, and the wire fixing portion may extend upward from a radially outer end of the extension portion. Therefore, the reel body and the wire fixing portion may rotate integrally.

Therefore, when the first driven gear 252a rotates, the reel body and the wire fixing portion may move the wire 230 by pushing or pulling the wire 230 while rotating together.

In addition, the second wire reel 253b may include a reel body formed in a cylindrical or circular column shape, a wire fixing portion disposed radially outward of the reel body and configured such that the wire 230 is fixedly coupled to the wire fixing portion, and a key portion protruding from an axially lower end of the reel body and coupled to a shaft portion of the second driven gear 252b. In this case, an axially lower end of the reel body may extend radially outward, the wire fixing portion may extend upward from a radially outer end of the extension portion, and the key portion may protrude and extend from an axially lower end surface of the reel body. Therefore, when the second driven gear 252b rotates, the key portion may rotate together with the second driven gear 252b, such that the reel body and the wire fixing portion may rotate. As a result, the second wire reel 253b may move the wire 230 by pushing or pulling the wire 230.

Meanwhile, the connecting part 250 may further include a support shaft 254. The support shaft 254 may be formed in a cylindrical shape, fixedly coupled to the connecting part housing 251, and configured to penetrate radial centers of the first driven gear 252a, the second driven gear 252b, the first wire reel 253a, and the second wire reel 253b. Therefore, the support shaft 254 may provide rotation axes of the first driven gear 252a, the second driven gear 252b, the first wire reel 253a, and the second wire reel 253b.

Therefore, according to the present disclosure, when the user manipulates (rotates) the first knob 121, the first driving sprocket 131 rotates together with the first knob 121, the first driven sprocket 141 rotates in conjunction with the first driving sprocket 131 by means of the first manipulation chain 151, and the first driving gear 171 rotates together with the first driven sprocket 141. Further, in the state in which the reusable part 100 and the disposable part 200 are coupled, the first driven gear 252a, which engages with the first driving gear 171, rotates in conjunction with the rotation of the first driving gear 171, and the first wire reel 253a rotates integrally with the first driven gear 252a, such that the wire 230 may be moved, and the insertion pipe 220 may be bent.

In addition, when the user manipulates (rotates) the second knob 122, the second driving sprocket 132 rotates together with the second knob 122, the second driven sprocket 142 rotates in conjunction with the second driving sprocket 132 by means of the second manipulation chain 152, and the second driving gear 172 rotates together with the second driven sprocket 142. Further, in the state in which the reusable part 100 and the disposable part 200 are coupled, the second driven gear 252b, which engages with the second driving gear 172, rotates in conjunction with the rotation of the second driving gear 172, and the second wire reel 253b rotates integrally with the second driven gear 252b, such that the wire 230 may be moved, and the insertion pipe 220 may be bent.

Meanwhile, in the case of a disposable endoscope in the related art, a biopsy channel and a supply channel are provided in a disposable part, and a biopsy channel and a supply channel, which are connected to the biopsy channel and the supply channel, are also provided in the reusable part. Therefore, contaminants may sometimes leak during a process of separating the disposable part and the reusable part.

In particular, because the biopsy channel and the supply channel are disposed adjacent to each other, contaminants in the biopsy channel are introduced into the supply channel when the insertion portion and the manipulation portion are separated, which may cause an inconvenience of having to wash the entire manipulation portion.

In order to solve this problem, in the present disclosure, the disposable suction part 300 may be further provided. The disposable suction part 300 may be detachably coupled to the reusable part 100 and accommodate therein a channel 320. In this regard, the disposable suction part 300 may be referred to as a component for an endoscope. That is, the component for an endoscope may be detachably coupled to the reusable part 100.

With reference to FIGS. 1 to 7, the disposable suction part 300 will be described below.

The disposable suction part 300 includes a suction part body 310, the channel 320, a suction valve 330, and a suction part handle 340.

The suction part body 310 may be detachably coupled to the reusable part housing 110. In this case, the suction part body 310 may be coupled to a surface of the reusable part housing 110 opposite to the surface of the reusable part housing 110 on which the manipulation knob 120 is disposed. Therefore, it is possible to prevent the manipulation knob 120 from interfering with the suction part body 310 when the manipulation knob 120 is manipulated. In addition, in the state in which the suction part body 310 is coupled to the reusable part housing 110, the user may hold the suction part body 310 and the reusable part housing 110 with one hand and manipulate the manipulation knob 120 with the other hand, such that the operator may stably grip the suction part body 310.

The suction part body 310 may include a first suction part body 311 and a second suction part body 312. One longitudinal side of the first suction part body 311 may be detachably coupled to the disposable part 200. In addition, the second suction part body 312 may be integrally connected to the other longitudinal side of the first suction part body 311 and extend in a direction intersecting a longitudinal direction of the first suction part body 311.

For example, the first suction part body 311 may be formed in a tubular shape opened partially in the longitudinal direction. Specifically, a portion of the first suction part body 311, which faces the reusable part housing 110, may be formed in an opened shape. With this configuration, in the state in which the suction part body 310 and the reusable part housing 110 are coupled, a space capable of accommodating the channel 320 may be formed in the first suction part body 311.

In this case, one longitudinal end of the first suction part body 311 may be formed in an opened shape, and a biopsy channel 321 may penetrate one longitudinal end of the first suction part body 311. In addition, at least a part of the biopsy channel 321 may be accommodated in the first suction part body 311. In addition, the other longitudinal end of the first suction part body 311 may be connected to the second suction part body 312.

Channel support ribs 311a may be formed in the first suction part body 311. The channel support rib 311a may protrude from an inner surface (inner peripheral surface) of the first suction part body 311 and be in contact with the biopsy channel 321, thereby supporting the biopsy channel 321. Therefore, the biopsy channel 321 may be stably supported while the reusable part 100 is used.

Meanwhile, the second suction part body 312 may be formed in a tubular shape opened partially in the longitudinal direction. Specifically, a portion of the second suction part body 312, which faces the reusable part housing 110, may be formed in an opened shape. With this configuration, in the state in which the suction part body 310 and the reusable part housing 110 are coupled, a space capable of accommodating the channel 320 may be formed in the second suction part body 312.

In this case, one longitudinal end of the second suction part body 312 may be connected to the first suction part body. In addition, at least a part of a suction channel 322 may be accommodated in the second suction part body 312. In addition, the other longitudinal end of the second suction part body 312 may be formed in an opened shape, and the suction channel 322 may pass through the other longitudinal end of the second suction part body 312.

The second suction part body 312 may have a channel fixing portion 312a. The channel fixing portion 312a may protrude from an inner surface (inner peripheral surface) of the second suction part body 312 and have a circular hole penetrated by the suction channel 322. Therefore, the suction channel 322 may be stably fixed while the reusable part 100 is used.

Meanwhile, the first suction part body 311 and the second suction part body 312 may be formed to have different inner diameters. In addition, heights by which the first suction part body 311 and the second suction part body 312 protrude from the reusable part housing 110 may be different based on the state in which the first suction part body 311 and the second suction part body 312 are coupled to the reusable part housing 110. For example, the first suction part body 311 may have a smaller diameter than the second suction part body 312. A height by which the first suction part body 311 protrudes from the reusable part housing 110 may be smaller than a height by which the second suction part body 312 protrudes from the reusable part housing 110. With this configuration, in the state in which the first suction part body 311 is coupled to the reusable part housing 110, a volume may be minimized, such that the operator does not feel discomfort when the operator grips the reusable part 100. In addition, the second suction part body 311 may ensure a space through which the supply channel 180 provided in the reusable part housing 110 may pass.

Meanwhile, the suction part body 310 may further have a coupling guide portion 313. The coupling guide portion 313 may extend from the first suction part body 311. In this case, the coupling guide portion 313 may be formed in a shape that surrounds at least a part of an outer side of an outer peripheral surface of the supply valve 185 based on the state in which the suction part body 310 is coupled to the reusable part housing 110. Therefore, the coupling guide portion 313 may guide the coupling so that the operator may intuitively recognize a coupled position when the operator couples the disposable suction part 300 to the reusable part 100.

The channel 320 is accommodated in the disposable suction part 300 and detachably coupled to the reusable part 100.

The channel 320 may provide a passageway through which a fluid passes. For example, the channel 320 may be formed in a cylindrical shape, and a gas, liquid, or biological material may pass through the inside of the channel 320.

The channel 320 includes the biopsy channel 321 and the suction channel 322. One side of the biopsy channel 321 may be connected to the suction valve 330, and the other side of the biopsy channel 321 may be detachably connected to the disposable part 200. The biopsy channel 321 may be connected to the biopsy channel 243 of the disposable part 200. Therefore, when a suction force is applied from a suction pump (not illustrated), the biological material introduced through the lighting/imaging part 210 may flow.

The suction channel 322 may connect the suction valve 330 and the suction pump (not illustrated). The suction channel 322 may transmit the suction force, which is applied from the suction pump (not illustrated), to the suction valve 330, and the air or the material having passed through the biopsy channel 321 may pass through the suction channel 322.

Meanwhile, the suction valve 330 may be disposed at a portion where the first suction part body 311 and the second suction part body 312 are connected to each other. The suction valve 330 may open or close the channel 320.

Specifically, the biopsy channel 321 and the suction channel 322 may be connected to the suction valve 330. That is, the biopsy channel 321 and the suction channel 322 may be connected to each other through the suction valve 330. Therefore, the operator may allow the biopsy channel 321 and the suction channel 322 to communicate with each other by operating the suction valve 330.

Specifically, in a state in which the suction valve 330 does not operate, the biopsy channel 321 and the suction channel 322 may not communicate with each other, and outside air may be sucked into the suction pump (not illustrated) through the suction channel 322.

Meanwhile, in case that the suction valve 330 operates, the biopsy channel 321 and the suction channel 322 may communicate with each other, the suction force of the suction pump (not illustrated) is applied to the suction channel 322 and the biopsy channel 321, and the biological material present at the periphery of the lighting/imaging part 210 may be sucked.

Meanwhile, the suction part handle 340 may be provided on the disposable suction part 300 of the present embodiment. The suction part handle 340 may protrude from an outer surface of the second suction part body 312. The suction part handle 340 may be provided such that the operator may grip the suction part handle 340. For example, the suction part handle 340 may be formed in a block or plate shape protruding by a predetermined height. In this case, a protrusion or a groove may be formed on the suction part handle 340 to increase a frictional force so that a finger of the operator does not slip.

Therefore, the operator may easily separate the disposable suction part 300 and the reusable part 100 by simply pulling the suction part handle 340.

A process of assembling the endoscope according to the embodiment of the present disclosure will be described with reference to FIG. 3.

First, the operator may couple the disposable suction part 300 to the reusable part 100. In this case, the operator may grip the suction part handle 340 and couple and fit the suction part body 310 to and with a channel seating portion 111. In this case, the operator may dispose the supply valve 185 between the coupling guide portion 313 and the suction valve 330 and dispose the suction channel 322 in a channel guide groove 111a. Further, when the operator presses the suction part body 310 toward the channel seating portion 111, a hook 314 may be coupled to the reusable part housing 110.

In the state in which the disposable suction part 300 and the reusable part 100 are coupled as described above, the coupling portion 190 may be inserted and coupled into the connecting part 250 of the disposable part 200. In this case, the operator may fit the guide pin with the groove of the connecting part 250 and fit and connect the biopsy channel 321 with and to the biopsy channel 241 of the disposable part 200. Further, the supply channel 180 may be connected to the supply channel 243 of the disposable part 200.

Therefore, according to the present disclosure, the operator may couple the disposable suction part 300 and the disposable part 200 by simply fitting the disposable suction part 300 and the disposable part 200 with the reusable part 100. Meanwhile, after the use of the endoscope 1 ends, the operator may pull and separate the disposable part 200 in the state in which the reusable part 100 and the disposable suction part 300 are coupled. In this case, the operator may release the hook 251a provided on the connecting part housing 251 and separate the connecting part housing 251 by pulling the connecting part housing 251 in a direction away from the reusable part 100. In this case, the channel 240 of the disposable part 200 may separate from the channel 320 of the disposable suction part 300 and the supply channel 180 of the reusable part 100.

Thereafter, the operator may grip the suction part handle 340 and separate the suction part handle 340 by pulling the suction part handle 340 in a direction away from the reusable part housing 110. Therefore, the channel 320, through which a biopsy material has flowed, may be separated from the reusable part 100 and separately discarded.

Therefore, according to the present disclosure, even though the endoscope 1 is used multiple times, only the biopsy channels 241 and 321, which are likely to be contaminated, may be separated and discarded, thereby improving the hygiene of the reusable part 100.

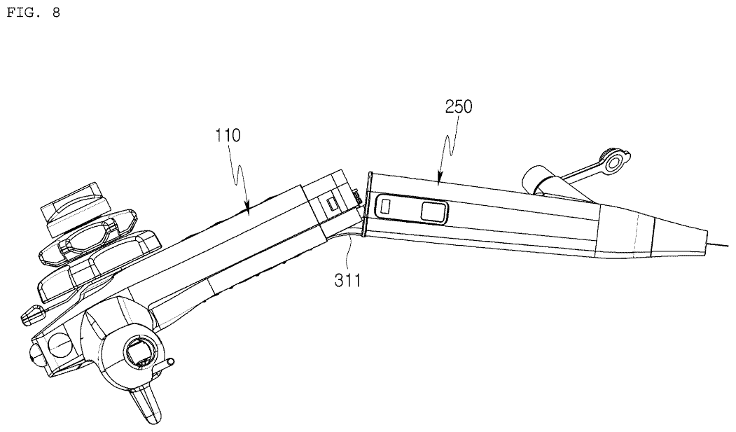

FIG. 8 illustrates an endoscope 1 according to a second embodiment of the present disclosure.

Meanwhile, to avoid the repeated description, the description of the endoscope 1 according to the first embodiment of the present disclosure may be applied, except for the components that have not been particularly described in the present embodiment, because the same structure and effect of the endoscope 1 may be applied.

The disposable suction part 300 of the present embodiment is characterized by being integrated with the disposable part 200.

In this case, the disposable suction part 300 and the disposable part 200 may be connected to be elastically transformed. Specifically, the first suction part body 311 of the disposable suction part 300 may be made of a material with elasticity. For example, the first suction part body 311 may be made of a material containing rubber.

Therefore, the first suction part body 311 may be elastically transformed.

Therefore, the operator may couple or separate only the reusable part 100 in the state in which the disposable suction part 300 and the disposable part 200 are integrated. During this process, the first suction part body 311 may protect and support the biopsy channel 321 therein while being elastically transformed.

Therefore, in the present embodiment, it is possible to prevent the biopsy channel, which is likely to be contaminated, from being separated from the disposable suction part 300 and the disposable part 200.

Therefore, according to the present disclosure, it is possible to prevent contaminants remaining in the biopsy channel from being introduced into the supply channel 180 provided in the reusable part 100.

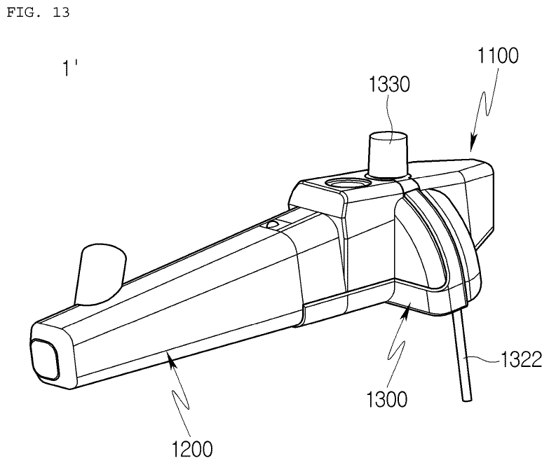

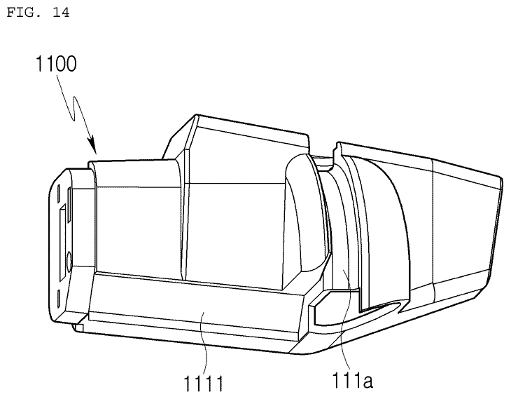

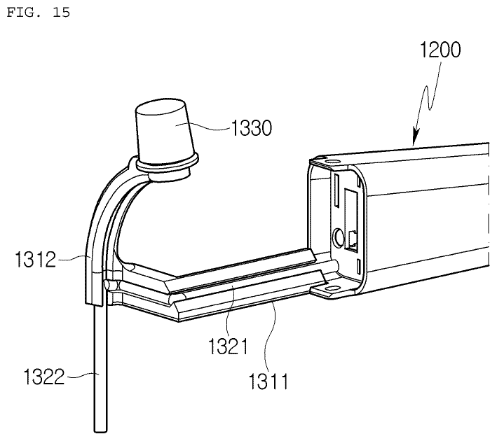

FIG. 13 is a perspective view for explaining an endoscope according to a third embodiment of the present disclosure, FIG. 14 is a perspective view for explaining a reusable part of the endoscope according to the third embodiment of the present disclosure, and FIG. 15 is a perspective view for explaining a disposable suction part of the endoscope according to the third embodiment of the present disclosure.

An endoscope 1′ according to a third embodiment of the present disclosure will be described below with reference to FIGS. 13 to 15.

Meanwhile, to avoid the repeated description, the description of the endoscope 1 according to the first embodiment of the present disclosure may be applied, except for the components that have not been particularly described in the present embodiment, because the same structure and effect of the endoscope 1 may be applied.

In a disposable suction part 1300 of the present embodiment, a first suction part body 1311 and a second suction part body 1312 may be made of an elastic material. For example, the first suction part body 1311 and the second suction part body 1312 may be made of a material containing rubber.

In this case, a biopsy channel 1321 may be accommodated in the first suction part body 1311. For example, a groove may be formed in the first suction part body 1311 and accommodate the biopsy channel 1321 in a longitudinal direction, and a diameter of the groove in a width direction may be equal to or finely smaller than a diameter of the biopsy channel 1321. In addition, a diameter of an outer end of the groove may be smaller than a maximum diameter. Therefore, when the biopsy channel 1321 is fitted into the groove, the first suction part body 1311 may tighten and support the biopsy channel 1321 by means of an elastic force.

In addition, a suction channel 1322 may be accommodated in the second suction part body 1312. For example, a groove may be formed in the second suction part body 1312 and accommodate the suction channel 1322 in a longitudinal direction, and a diameter of the groove in a width direction may be equal to or finely smaller than a diameter of the suction channel 1322. In addition, a diameter of an outer end of the groove may be smaller than a maximum diameter. Therefore, when the suction channel 1322 is fitted into the groove, the second suction part body 1312 may tighten and support the suction channel 1322 by means of an elastic force.

Meanwhile, in the present embodiment, the biopsy channel 1321 and the suction channel 1322 may be connected. Further, a suction valve 1330 may be connected to one end of the suction channel 1322.

Therefore, when the operator operates the suction valve 1330, the biopsy channel 1321 and the suction channel 1322 may communicate with each other, and the suction force may be applied to the biopsy channel 1321.

Meanwhile, the channel seating portion 1111 is formed on a reusable part housing 1110 so that a suction part body 1310 may be fitted, and the channel guide groove 111a, to which the channel is coupled, may be formed. In this case, because the suction part body 1310 is made of an elastic material, the suction part body 1310 is elastically transformed while being fitted with the suction part body 1310, and then the suction part body 1310 may be supported by the reusable part housing 1110 while being restored.

With this configuration, the operator may easily couple a reusable part 1100 and the disposable suction part 1300.

Meanwhile, in the present embodiment, the disposable suction part 1300 and a disposable part 1200 may be integrated. Therefore, the operator may couple the disposable suction part 1300 while coupling the disposable part 1200 and the reusable part 1100.

As described above, according to the endoscope according to the present disclosure, the operator may couple the disposable part and the disposable suction part by simply fitting the disposable part and the disposable suction part with the reusable part.

In addition, after the disposable part is used, the operator may separate the disposable suction part from the reusable part and separately discard the disposable suction part, thereby improving the hygiene of the reusable part.

In addition, because the disposable part and the disposable suction part are integrated, it is possible to prevent contaminants remaining in the biopsy channel from being introduced into the supply channel provided in the reusable part after the disposable part is used.

While the present disclosure has been described with reference to the specific embodiments, the specific embodiments are only for specifically explaining the present disclosure, and the present disclosure is not limited to the specific embodiments. It is apparent that the present disclosure may be modified or altered by those skilled in the art without departing from the technical spirit of the present disclosure.

All the simple modifications or alterations to the present disclosure fall within the scope of the present disclosure, and the specific protection scope of the present disclosure will be defined by the appended claims.

What is claimed is:

1. An endoscope comprising:
a disposable part having a lighting/imaging part and an insertion pipe;
a reusable part detachably coupled to the disposable part, having a manipulation knob, and configured to bend the insertion pipe by means of a manipulation of the manipulation knob; and
a disposable suction part detachably coupled to the reusable part and configured to accommodate therein a channel.

2. The endoscope of claim 1, wherein the disposable suction part has a suction valve configured to open or close the channel.

3. The endoscope of claim 2, wherein the channel comprises:
a biopsy channel having one side connected to the suction valve, and the other side detachably connected to the disposable part; and
a suction channel configured to connect the suction valve and a suction pump.

4. The endoscope of claim 1, wherein the reusable part accommodates a supply channel in which air or liquid flows.

5. The endoscope of claim 1, wherein the disposable suction part comprises:
a first suction part body having one longitudinal side detachably coupled to the disposable part; and
a second suction part body extending in a direction intersecting a longitudinal direction of the first suction part body.

6. The endoscope of claim 5, wherein the disposable suction part further comprises a suction part handle protruding from an outer surface of the second suction part body.

7. The endoscope of claim 1, wherein the disposable part is coupled in a state in which the disposable suction part and the reusable part are coupled.

8. The endoscope of claim 1, wherein the disposable suction part and the disposable part are integrated.

9. The endoscope of claim 8, wherein the disposable suction part and the disposable part are connected to be elastically transformable.

10. The endoscope of claim 1, wherein the reusable part comprises a grip guide portion provided on an outer surface and configured to guide a grip position.

11. A component for an endoscope, which is detachably coupled to a reusable part having a manipulation knob, the component comprising:
a first suction part body configured to accommodate therein a biopsy channel; and
a second suction part body configured to accommodate therein a suction channel and extending in a direction intersecting a longitudinal direction of the first suction part body.

12. The component of claim 11, further comprising:
a suction part handle protruding from an outer surface of the second suction part body and configured to be gripped.

13. The component of claim 11, further comprising:
a suction valve configured to operate to allow the biopsy channel and the suction channel to communicate with each other.

14. The component of claim 11, further comprising:
a disposable part having a lighting/imaging part and an insertion pipe,
wherein the first suction part body integrated with the disposable part.

15. The component of claim 14, wherein the first suction part body is made of an elastic material.
